(19) United States
(12) Patent Application Publication
(10) Pub. No.: US 2022/0119669 A1
(43) Pub. Date: Apr. 21, 2022

(54) GLOVES WITH DURABLE LUBRICITY AND IMPROVED WET DONNING, AND METHODS THEREOF

(71) Applicant: HydroGlyde Coatings LLC, Brookline, MA (US)

(72) Inventors: Stacy Lee Chin, Brookline, MA (US); Benjamin Goldman Cooper, Lynnfield, MA (US)

(21) Appl. No.: 17/268,475

(22) PCT Filed: Sep. 17, 2019
(86) PCT No.: PCT/US19/51436

§ 371 Date: Feb. 14, 2021

Related U.S. Application Data
(60) Provisional application No. 62/735,971, filed on Sep. 25, 2018.

(51) Int. Cl. C09D 139/06; A41D 19/00; B29C 41/14; B29C 41/22; C09D 121/02; C08J 5/02; C08J 7/056; A61B 42/10

(52) U.S. Cl. CPC C09D 139/06; A41D 19/0006; A41D 19/0058; A41D 19/0082; B29C 41/14; B29K 2007/00; C09D 121/02; C08J 5/02; C08J 7/056; A61B 42/10; B29C 41/22

(57) ABSTRACT

The invention provides a novel hydrophilic polymer and rubber polymer microparticles blend coating formulation and compositions thereof for rubber gloves to improve wet donning properties for easy application. More particularly, the invention relates to a novel hydrophilic polymer and rubber polymer microparticles blend coating formulation and compositions thereof, and their use on gloves to form hydrophilic and flexible coatings with durable lubricity. The gloves of the invention exhibit good donnability, tensile strength, elongation to break, and stress at 500% elongation.

### PRIORITY CLAIMS AND RELATED PATENT APPLICATIONS

This application claims the benefit of priority from U.S. Provisional Application Ser. No. 62/735,971, filed on Sep. 25, 2018, the entire content of which is incorporated herein by reference in its entirety.

### TECHNICAL FIELD OF THE INVENTION

The invention generally relates to polymer formulations and use thereof in surface coatings for rubber gloves. More particularly, the invention relates to a novel hydrophilic polymer and rubber polymer blend coating formulation and compositions thereof, and their use on rubber gloves to form hydrophilic and flexible coatings with durable lubricity and improved donning properties.

### BACKGROUND OF THE INVENTION

The material surface properties of rubber are of significant importance for medical devices and tools (e.g., gloves) in research, healthcare, and medicine. In addition to their soft, flexible, and durable features, rubbers exhibit ideal chemical, physical, mechanical, and rheological properties, such as low heat- and water-resistance, elasticity, and chemical resistance to polar organic solvents. Rubber also exhibits numerous advantages as the constituent material demonstrates excellent barrier properties, low manufacturing cost, and facile processability. Common commercial products made with rubber include those with natural (latex), neoprene, nitrile, and butadiene-based synthetic rubber-based materials.

A primary objective for healthcare and research environments is to protect individuals from exposure from any hazardous biologics or materials through the practice of utilizing proper protection equipment (PPE). Rubber gloves are critical to protect healthcare workers and patients from exposure of any healthcare-associated infections or exposure to communicable diseases. In a research laboratory environment, researchers and technicians are required to wear disposable gloves when working with chemicals and other like materials to protect hands and skin from exposure to hazardous chemicals, contaminations, infectious diseases, therapeutics, and biologics.

However, due to the soft, hydrophobic nature of rubber, the material typically exhibits high frictional properties when in contact with another surface, such as skin, as rubber has a low elastic, modulus with a tendency to be “sticky” or “grippe” to other surfaces. This is evident when fluids or moisture is present between the two surfaces. For instance, individuals in healthcare, research, and food preparation encounter challenges with the process of putting on and removing gloves due to poor their donning properties especially in the presence of moisture from sweat or washing of the hands. In return, exposing rubber materials to moist hands (e.g., sweat or after washing hands) and at elevated temperature due to body heat can result in an uncomfortable, burdensome experience for users. For example, surgeons are required to wash their hands with soap and water for sterilization prior towards the donning of surgical gloves. This process has proven to be challenging for surgeons as wet hands inhibits the ease of glove donning. In another instance, researchers or laboratory technicians who wear disposal rubber gloves for long periods of time most likely generate moisture due to sweat between the glove and skin interface. A common solution to this issue includes frequent glove changes to prevent exposure to prolong moisture. However not only does this process inhibit productivity but presents a challenge for the wearer to remove the used glove and put a new one.

There remain diverse challenges in manufacturing rubber gloves with improved quality and productivity to address problems with donning.

### SUMMARY OF THE INVENTION

The invention is based in part on the unexpected discovery of a novel blend coating formulation of hydrophilic polymer and rubber polymer microparticles (referred to herein as HPRB) and compositions thereof, that are suitable for application on rubber surfaces, particularly on gloves, to improve, among other things, the ease of donning, the process of putting on and taking off disposal gloves for users, and usability with respect to mitigating sticking or restriction of motion when in presence of moisture, aqueous fluids, fluids, liquids, water or like-substances.

In one aspect, the invention generally relates to a fluid composition, which includes: a hydrophilic polymer and a suspension of rubber polymer microparticles in solvent or suspending fluid, and, optionally, one or more of vulcanizing or accelerating agents including stabilizers, a crosslinker, a vulcanization activator, a vulcanization accelerator, an antioxidant, an antiozonant and optionally, white or other colored pigments.

The hydrophilic polymer has a mean molecular weight in the range from about 1 kDa to about 300,000 kDa and is present in the composition at a concentration from about 0.1 w/v % to about 10 w/v %. The rubber polymer microparticles are present in the composition at a concentration from about 10 w/v % to about 60 w/v %. The weight ratio of the hydrophilic polymer to the rubber polymer microparticles is in the range from about 1:1 to about 10:1.

In certain embodiments, the HPRB formulation includes a high molecular weight hydrophilic polymer in the rubber suspension which includes poly(N-isopropylacrylamide) (PNIPAM), polyacrylamide (PAM), poly(-oxazoline) and polyethylenimine (PEI), poly(acrylic acid), polymethacrylate and other acrylic polymers, poly(ethylene glycol) and poly(ethylene oxide), poly(vinyl alcohol) (PVA) and copolymers, poly(vinylpyrrolidone) (PVP) and copolymers, polyelectrolyte, cucurbit[n]uril hydrate, as well as co-block or tri-block polymers.

In certain embodiments, there are more than one hydrophilic polymer present in the resulting HPRB suspension formulation. In certain preferred embodiment, the high molecular weight hydrophilic polymer is PVA, PEG, or PVP. In certain preferred embodiments, the high molecular weight hydrophilic polymer is PVP.

In certain preferred embodiments, the hydrophilic polymer(s) is first to be dissolved in aqueous solution to facilitate blending with the rubber suspension.

In certain embodiments, the HPRB formulation includes a combination of a rubber suspension, a blend of a secondary rubber suspension, stabilizers, crosslinkers, vulcanization accelerators, hydrophilic polymers vulcanization activators, antioxidants, antiozoant, or colored pigment. The above rubber dispersion in solution can be included in solution as dry or active parts of 0 to about 100 w/v % whereas the other ingredients can be added in solution as dry or active parts from a rage of about 0.01 to about 10 w/v %.

In another aspect, the invention generally relates to a composition formed by mixing: a first aqueous solution of a hydrophilic polymer with a concentration in the range from about 0.1 w/v % to about 10 w/v %; and a second aqueous suspension of rubber polymer microparticles with a concentration in the range from about 10 w/v % to about 60 w/v %. The hydrophilic polymer has a mean molecular weight in the range from about 1 kDa to about 1,000 kDa. The volume ratio of the first aqueous solution to the second aqueous suspension is in the range from about 1:1 to about 1:3.

In yet another aspect, the invention generally relates to a cured material formed by heating a composition disclosed herein for a time sufficient to form interpenetrating polymer networks of crosslinked hydrophilic polymer and rubber polymer which is facilitied via the reagents in the blend. The process results in the physical entrapment of the hydrophilic polymer on the rubber surface upon the heat curing process. Interpenetrating polymer networks can reinforce and improve the properties of the resulting polymer matrix. This curing process can also include but is not limited to using chemical reagents to form the polymer network.

The coated rubber gloves are manufactured in accordance to the present invention may follow accordingly. A former, usually composed of porcelain or glass, in a contoured shape of a hand or glove is first dried using heat which can be done in an oven which is followed by a dipping in an alcohol-, water-, acid-, or coagulant-based dispersion which may include calcium nitrate, calcium carbonate, cornstarch, or wetting agent. This layer on the former is then exposed to heat to be dried which is then followed by a secondary dip into a rubber or synthetic elastomer compound or formulation dispersion. The second dip may occur while the first layer is still at above-ambient temperature to facilitate polymer interpenetration due to the more permeable melted state at elevated temperature, or it may be performed at ambient temperature. This following the similar heat coagulation process. This step can be repeated multiple times to result in a thicker glove. The preferred is to repeat the dipping and coagulating process twice. Finally, the former with the desired glove is removed from the oven and stripped from the former. In a preferable aspect, prior to the removal of the glove, the former with the final coated glove undergoes a leaching process prior to vulcanization to remove any residual calcium nitrate or soluble protein. The leaching process which the glove former can undergo may include but is not limited to exposure to hot water or an aqueous acidic solution.

In certain embodiments, the rubber coating formulation can be alternatively applied to the inner surface of the glove, after completion of the chlorination process of either the outer or inner glove surface in an on-line or after-processed step upon completion of oven drying. The purpose of the post coating application process is to optimize the glove donning properties without the use of powders for both wet and dry donning applications. Additional coatings can also be added upon the rubber layer with the hydrophilic coating which can include polyacrylates, polyurethanes, combinations of the two, hydrogels, and nonhydrogels. Primers can be applied to improve the adhesion of the coating properties which can be applied as an overdip to improve binding of the coated material.

In certain embodiments, rubber blends with the hydrophilic polymer can also include other suitable lubricating reagents to further enhance the gloves donning properties. These reagents include surfactants, nonionic and ionic surfactants. In certain embodiments, the rubber layer includes an emollient coating layer of the hydrophilic polymer in addition with glycerol, gluconolactone, D-sorbitol, provitamin-B and chitosan.

In certain embodiments, the hydrophilic-coated polymer is included in the portion of the rubber layer intended to be in contact with the skin.

In certain embodiments, the final HPRB coated glove undergoes a post processing chlorination step. This process can include treating the glove with acid, chemicals, chlorination, or oxidation processes to improve its donning characteristics. Other methods can also include exposure of the final gloves to an aqueous chlorine solution for about 1 to about 10 minutes, or spraying and tumbling of an additional coating or emulsions on the inner rubber layer with the hydrophilic polymer blend. For example, the resulting coated glove can undergo a chlorination process either outside or, preferably inside, of the coated rubber glove. This procedure exposes chlorine to the glove surface which may smooth the rubber surface or further enhances its donning properties. Other techniques which may be also considered are online chlorination post-dip, chlorination post processing, or halogenation which can be performed to improve the glove donning and its physical properties.

In certain embodiments, the HPRB coated glove undergoes a leaching processing step. In some embodiment, this manufacturing process exposes the final HPRB coated glove up to 160 degrees and an aqueous solution to extract proteins and other residuals from the latex. In other aspects, this process can occur before or after the final HPRB application onto the glove.

In certain embodiments, the HPRB coated glove undergoes a beading process to further enhance the donning and stripping process of the final product. Beading can be done to improve vulcanization, durability, and chemical resistance of the final product. In other aspects, this process can occur before or after the final HPRB application onto the glove.

In certain embodiments, the HPRB coated glove undergoes a wet powdering process called a slurry to apply a powder which further enhances glove donning. In other aspects, this process can occur before or after the final HPRB application onto the glove. In other aspects, this process is followed by an oven exposure to heat between about 100° C. to about 250° C. for a period of 0 to about 10 minutes.

In certain embodiments, the HPRB coated glove undergoes a dry slurry process.

In certain embodiments, the HPRB coated glove undergoes a tumbling process to apply the slurry which can be composed of starches and/or biocides.

In certain embodiments, the HPRB coated glove undergoes a stripping process.

In certain embodiments, the HPRB coating is applied to the outside of the glove for easier application which can be turned inside out during the stripping process.

In certain embodiments, the HPRB coated glove undergoes sterilization. In certain embodiments, the sterilization procedure includes one or more of ethylene oxide sterilization, autoclave, radiation, gamma, or electron beam radiation sterilization.

In certain embodiments, the final glove includes an intermediate layer of a rubber blend of natural or synthetic rubber interposed between the elastomeric glove and the coating inner rubber layer. Upon the polymer coated glove, additional donning or moisturizing reagents can be incorporated which can include but is not limited to potato starch, lycopodium, cornstarch, aloe vera, lotions, creams, and vitamins.

In certain embodiments, the HPRB coated rubber glove is embedded with other polymer or agents used to ease donning. In another embodiment, the resulting polymer coated gloves can have an additional layer of other donning reagents such as aloe vera, silicone, other polymer coatings, cornstarch, or blends of the sort. In another embodiment, the resulting polymer coating can be a layer on top of these current existing donning reagents to further enhance the glove's donning properties under wet and dry environments.

In certain embodiments, the glove involves applying multiple layers of the hydrophilic and rubber polymer blend coating formulation over a rubber base layer, which is applied to the rubber product surface.

In some embodiments, the sequential hydrophilic coating application and curing process may be repeated a plurality of times. For example, a hydrophilic and rubber polymer coating is applied onto a rubber substrate with a subsequent layer being the hydrophilic and rubber polymer rubber.

In certain embodiments, the subsequent hydrophilic and rubber polymer is applied onto a hydrophilic and rubber polymer coating blend that served as the first or base layer.

In certain embodiments, multiple coatings are applied dependent on the desired properties to be achieved. To improve durability of the coating, it may be necessary to form a gradient with high rubber-to-hydrophilic polymer ratio proximal to the basal glove layer, and a low rubber-to-hydrophilic polymer ratio distal to the basal glove (e.g., at the composite surface), ensuring maximal adherence to the base layer and maximal hydrophilicity at the surface).

A key feature of the invention is that the inner-coated layer of the glove is a cross-linked polymer blend. When in contact with moisture, aqueous fluids, fluids, liquids, water or like-substances, the inventive hydrophilic coating becomes slippery with durable lubricity, resulting in lowered frictional forces when in contact with tissue or other interfaces. This thin hydrophilic coating is able to maintain its lubricity continuously when in contact with surfaces in the presence of water without impacting on the mechanical and physical properties in order to improve its wettability and donning properties, or improve the user's ability to put on and remove gloves.

In certain embodiments, the rubber blend and multiple layers include more than one different rubbers in dispersion in presence of the hydrophilic polymer which can be a combination of the two following rubbers: natural rubber latex (NRL), neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, other butadiene-based synthetic rubber-based materials, other styrene diblock and triblock copolymers, or other synthetic elastomers. The glove feature can be made with multiple different rubber layers where the hydrophilic polymer is included in the rubber layer within the inside of the glove. This can include but is not limited to top layer to be natural rubber latex (NRL), neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, and other butadiene-based synthetic rubber-based materials whereas the inner layer can be the same or be any of the other rubbers that includes the hydrophilic polymers.

In certain embodiments, the HPRB coating is applied to the entire surface of the inner glove, portions of the glove, the outer side of the glove. In certain preferred embodiments, the coating is to be applied to the inner side of the glove, whether it is the entire surface or portions of the inner glove with the intention to be in contact with skin.

In another aspect, the invention generally relates to a method for manufacturing a glove. The method includes: cleaning one or more glove glass or porcelain formers; dipping the one or more glove glass or porcelain formers into a coagulant barrel; dipping the one or more glove glass or porcelain formers into a latex barrel; heating the coated one or more glove glass or porcelain formers; second dipping of the coated one or more glove glass or porcelain formers into the coagulant barrel; second dipping of the coated one or more glove glass or porcelain formers into the latex barrel; second heating of the coated one or more glove glass or porcelain formers; third dipping of the coated one or more glove glass or porcelain formers into a barrel with the composition disclosed herein; third heating of the coated one or more glove glass or porcelain formers to cure the hydrophilic polymer and rubber polymer; beading and vulcanizing the gloves with heat; exposing the gloves to a carbonate slurry; drying the gloves with heat; and stripping to obtain the manufactured gloves.

In yet another aspect, the invention generally relates to a glove manufactured according to a method disclosed herein.

Any rubber disposal glove may be manufactured accordingly, for example, surgeon's gloves, microsurgery gloves, dental surgeon's gloves, orthopedic surgeon's gloves, autopsy surgeon's gloves, specialty/chemotherapy surgeon's gloves, radiation attenuating surgeon's gloves, non-medical gloves, embalming gloves, food service gloves, cleaning gloves, examination gloves, rubber gloves for research, and finger cots.

A purpose of the invention is to improve wet donning of rubber disposable gloves. Another purpose of the invention is to improve dry donning of rubber disposable gloves. Another purpose of the invention is to improve donning under wet conditions, such as after washing hands or due to prolong moisture (e.g., sweat) on hands.

### BRIEF DESCRIPTION OF THE DRAWINGS

FIG. 1. Scanning electron microscopy (SEM) images taken at 500× magnification of (A.) non-coated gloves; (B.) commercially available polymer coated glows (bioggel); (C.) nitrile-coated gloves; (D.) powdered coated glove; and, (E.) polymer coated gloves.

### DEFINITIONS

Unless stated otherwise, or implicit from context, the following terms and phrases include the meanings provided below. The definitions are provided to aid in describing particular embodiments, and are not intended to limit the claimed invention, because the scope of the invention is limited only by the claims.

The term “about” when used in connection with a value can mean 5% of the value being referred to. For example, “about 100” refers to a value in the range of 95 to 105.

As used herein, the term “latex” refers to natural or synthetic latex, which include vulcanized or non-vulcanized. The term “latex polymer” refers to the polymer(s) the latex is formed from. Typically, the latex substrate is hydrophobic. In some embodiments, the latex is non-cytotoxic and/or biocompatible provided that the user does not have an adverse or allergic reaction when in contact with latex. Synthetic latex may include synthetic rubber materials, including nitrile, hydrogenated nitrile, ethylene-propylene, fluorocarbon, chloroprene, silicone, fluorosilicone, polyacrylate, ethylene acrylic, acrylic polymers, styrenebutadiene, acrylonitrile butadiene, polyvinyl acetate, or polyurethane rubbers.

As used herein, the term “rubber” refers to the elastomeric material which may or may not include vulcanized or non-vulcanized. Typically, the rubber substrate is hydrophobic. The term rubber may compose of the following or a blend of the following items: natural rubber latex (NRL), synthetic latex, neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, and other butadiene-based synthetic rubber-based materials.

As used herein, the term “hydrophilic polymer” refers to homo- or co-polymers that exhibit hydrophilic properties, i.e., having a strong affinity for water. Non-limiting examples of hydrophilic polymers include poly(vinyl pyrrolidone)(PVP), poly(ethylene glycol) (PEG), poly(vinyl alcohol) (PVA), poly(N-isopropylacrylamide), polyacrylamide, poly(-oxazoline), polyethylenimine, polyacrylic acid), polymethacrylate, poly(-ethylacrylic acid), poly(acrylic acid), poly(sulfopropyl acrylate) potassium salt, poly(-methacryloyloxyethyl phosphorylchlorine), poly(-propylacrylic acid), poly(methacrylic acid), poly(-hydroxypropyl methacrylate), hydroxypropylmethylcellulose (HPMC), poly(oxanorbornene), polyelectrolytes, and co-polymers thereof.

As used herein, the term “hydrophilic latex blend” or “hydrophilic and latex polymer blend” refers to an evenly-mixed and viscous solution mixture composed of a hydrophilic polymer and latex dissolved in an aqueous solution.

As used herein, the acronym “HPRB” references to the hydrophilic polymer and rubber polymer blend coating formulation and compositions to be applied and used for rubber glove coatings. A purpose of the intended coating invention is to be applied to the rubber gloves to improve donning or easy of putting on or taking off rubber disposable gloves for the user.

As used herein, the term “non-cytotoxic” refers to biocompatibility with mammalian cells.

As used herein, the term “biocompatible” refers to the absence of an adverse acute, chronic or escalating biological response to an implant or coating, and is distinguished from a mild, transient inflammation which typically accompanies surgery or implantation of foreign objects into a living organism.

As used herein, the term “viscous” refers to a liquid material, e.g., a solution having viscosity of several hundred centipoises to several million centipoises. For example, the measurement of viscosity can range from about 10² cP to about 10⁷ cP.

### DETAILED DESCRIPTION OF THE INVENTION

The invention provides a novel blend coating formulation of hydrophilic polymer and rubber polymer microparticles and compositions thereof, that are suitable for application on rubber surfaces, particularly on gloves, to improve, among other things, the ease of donning when in presence of moisture, aqueous fluids, fluids, liquids, water or like-substances, the process of putting on and taking off disposal gloves for users, and usability with respect to mitigating sticking or restriction of motion.

The HPRB coating of the invention exhibits improved (1) adherence to the rubber with no cracking or delamination, (2) durability and donning characteristics, and (3) tensile strength and elasticity. The HPRB coating can be integrated and applied onto different rubber materials including natural rubber latex (NRL), neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, other butadiene-based synthetic rubber-based materials, other styrene di-block and tri-block copolymers, or other synthetic elastomers, including blends thereof.

Importantly, the HPRB coating invention is intended to be applied onto an inner surface of a rubber disposal glove to improve donning and usability with respect to interaction with the user's hand. In other applications, the HPRB coating may be applied to the outer surface of gloves to afford an interaction with non-user objects and tissues that is, among other properties, lubricious, non-sticking, gliding, shear-force-reducing, and/or non-trauma-inducing. The HPRB coating includes a non-cytotoxic and/or biocompatible, cross-linked rubber blended with the hydrophilic polymer coating which may be applied onto the material during the on-line glove manufacturing processes, in an off-line process off the manufacturing line, or afterwards in a post-processing step.

Since the purpose of standard disposable gloves in research and healthcare is to provide a non-permeable barrier that safely protects hands in work environments, this actually can have a negative impact for the user due to prolonged usage. For example in severe circumstances, the prolonged exposure of moisture or sweat to rubber can lead to skin problems or even weaken the rubber material, thus making it more vulnerable to become brittle or break. Additionally, this can also result in a lack of proper airflow, increased frictional forces between the glove and skin due to repeated wear, and the creation of elevated temperatures within the glove which increases perspiration and discomfort for the wearer. Additionally, the moist environment between the glove and skin can also create an ideal breeding ground to promote bacterial and fungal growth. These undesirable side effects can expose glove users to more hazardous risks within their working environment. Thus, it is critical to improve the donning of rubber gloves in order to improve users' dexterity, tactility, comfort and mobility associated with proper usage of gloves for optimal and proper protection and safety.

To address these challenges, a number of solutions have been developed through the application of apply creams, powder, gels, lubricants, or emollients as an additive layer to protect hands and ease the glove application and donning processes, with dry or wet hands. A common donning reagent is to add powder to latex gloves in the manufacturing process as a way to facilitate users to put on and remove gloves. However, due to known hazards associated with skin irritation and inhalation of these powders, the FDA highly advises to resort towards other alternative donning approaches and encourages the practice of using powder-free gloves. In response, new polymer coating technologies have emerged to replace the use of powder as a donning lubricant in the manufacturing of surgical, examination, and research rubber gloves. These polymer coatings are typically applied at the end of the glove manufacturing process inside of the glove and must meet the following requirements:
(1) good adherence to the rubber surface without any cracking, delamination, or leaching
(2) good durability and donning characteristics, as well as good tensile strength and elasticity
(3) resistant to chlorination or any post-forming processes in the glove manufacturing processes, and
(4) no degradation upon sterilization by autoclaving, radiation, or other forms of sterilization.

Research strategies to modify the chemical, physical, and mechanical properties of rubber are of significant research and commercial interest as a means to: (1) reduce frictional forces between rubber surface and tissue interfaces; (2) minimize protein adsorption and platelet adhesion to afford non-fouling surfaces on rubber substrates; and, (3) improve the blood-compatibility of rubber-based materials to expand their application in medical devices. However, rubber has proven to be challenging to modify and coat due to its flexibility and the limited functional groups for chemical modifications. Attempts were made to introduce hydrophilic coatings on the surface, or perform surface modification reactions using peroxides or light to attach hydrophilic small molecules or low molecular weight polymers on rubber substrates. However, these efforts frequently resulted in:
(1) the need to use expensive starting materials and harsh chemical reagents for surface modification reactions under strict conditions,
(2) poor coating adhesion with cracking or delamination on the rubber surface,
(3) low grafting yields of hydrophilic small molecules,
(4) inflicted damage on rubber substrates, and
(5) lengthy procedures to pretreat surface (e.g., argon plasma, ozone) to improve grafting yield.

Due to these technical challenges, there are a limited number of polymer coatings compatible for rubber gloves to facilitate the donning process or wearers to put on and take off rubber gloves, particularly when in presence of moisture, aqueous fluids, fluids, liquids, water or like-substances. The ability to apply polymer coatings on rubber is highly influenced by it ability to bond onto the rubber substrate while exhibiting good durability and elasticity. Poor bonding of the polymer coating can result in delamination or cracking during processing or donning. To improve adhesion and durability of these polymer coatings onto gloves, a number of additional treatment processes has to be performed including the application of hydrogels, polyurethanes, chlorinated reagents, acrylic, silicone, or nitrile coatings in the inner surface of rubber gloves. However, these additional processes increase costs for the manufacturer and these current solutions do not even offer a viable solution for disposable glove users.

Therefore, novel methods are desired to introduce hydrophilic, non-fouling, and lubricious surfaces on natural rubber latex (NRL), neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, and other butadiene-based synthetic rubber-based materials to increase the compatibility to apply polymer coating onto rubber surfaces for gloves and other applications. In return, this initiative can also expand rubber's current repertoire towards new products in the commercial markets.

The invention provides a novel hydrophilic polymer and rubber polymer blend coating formulation, compositions thereof, and its application onto disposable rubber gloves. The purpose of the invention is to provide an innovative polymer coating that enables the easy application to adhere onto a variety of different rubber elastomeric compounds which becomes slippery and lubricated when in the presence of fluids or moisture. The HPRB coating is intended to improve, among other things, the ease of (1) donning, (2) the process of putting on and taking off disposal gloves for users, and (3) usability with respect to mitigating sticking or restriction of motion. The invention herein offers a coating with good (1) adherence to the rubber with no cracking or delamination; (2) durability and donning characteristics; and (3) tensile strength and elasticity.

The HPRB coating formulation includes a hydrophilic high molecular weight polymer (e.g., poly(vinyl pyrrolidone)(PVP), poly(ethylene glycol) (PEG), or poly(vinyl alcohol) (PVA) that is blended and integrated with a rubber suspension (e.g., having a similar or like composition as the elastomeric substrate to be coated) in solution with the presence of accelerators, oxidants, preservatives. The rubber suspension in solution can include one or more of the following: natural rubber latex (NRL), neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, other butadiene-based synthetic rubber-based materials, other styrene di-block and tri-block copolymers, or other synthetic elastomers, including blends thereof. Additionally, the HPRB formulation is preferably a non-cytotoxic and/or biocompatible, cross-linked rubber blended with the hydrophilic polymer coating which is applied onto the material during the on-line glove manufacturing processes or afterwards in a post-processing step. These two components are present at a pre-selected ratio and mixed so as to generate a homogenous and viscous solution (a liquid suspension). In a typical coating application, for example to a former, typically made of glass or porcelain, a thin and even layer of an uncured rubber suspension solution is applied to the surface of former (either after a base layer is formed or directly on the latex glove) via dip-coating, followed by exposure to heat to evaporate the solvent and form a cured coating. Typically, upon completion of this process, the HPRB coated rubber glove can also undergo additional post-processing, sterilization, or chlorination steps.

The invention generally relates to a method for manufacturing a rubber disposable glove. The method includes: providing a sheath of an elastomeric material selected from natural or synthetic rubber latex, the sheath having an outer surface and an inner surface; depositing a first layer of an aqueous suspension of a rubber polymer to at least a portion of the outer surface of the sheath; curing the rubber polymer with exposure to heat for a time sufficient to form a first or base layer of cured rubber polymer; depositing a second layer of a composition, comprising a hydrophilic polymer and a suspension of rubber polymer, to at least a portion of the first or base layer; and curing the second layer of hydrophilic polymer and latex polymer with exposure to heat for a time sufficient to form a second or top layer. The hydrophilic polymer has a mean molecular weight in the range from about 1 kDa to about 300,000 kDa and is present in the composition at a concentration from about 2 w/v % to about 10 w/v %. The latex polymer microparticles are present in the composition at a concentration from about 20 w/v % to about 65 w/v %. The weight ratio of the hydrophilic polymer to the rubber polymer microparticles is in the range from about 1:1 to about 10:1.

Importantly, the HPRB coating invention is intended to be applied onto the inner surface of a rubber disposal glove to improve donning and usability with respect to interaction with the user's hand. In other applications, the HPRB coating may be applied to the outer surface of gloves to afford an interaction with non-user objects and tissues that is, among other properties, lubricious, non-sticking, gliding, shear-force-reducing, and/or non-trauma-inducing. When the HPRB coating is in contact with water, aqueous solutions, or fluids, the hydrophilic coating becomes durably lubricious. This thin hydrophilic coating is able to maintain its lubricity continuously when in contact with surfaces in the presence of water without impacting on the mechanical and physical properties of the coated rubber material. Thus, frictional forces are lowered when in contact with tissue or other interfaces within the process of putting on or removing gloves.

In one aspect, the invention generally relates to an aqueous composition, which includes: a hydrophilic polymer and a suspension of rubber polymer microparticles. The hydrophilic polymer has a mean molecular weight in the range from about 1 kDa to about 300,000 kDa and is present in the composition at a concentration from about 0.1 w/v % to about 10 w/v %. The rubber polymer microparticles are present in the composition at a concentration from about 20 w/v % to about 60 w/v %. The weight ratio of the hydrophilic polymer to the latex polymer microparticles is in the range from about 1:1 to about 10:1.

Any suitable hydrophilic polymer may be employed. In certain embodiments, the hydrophilic polymer includes one or more hydrophilic polymers selected from the group consisting of: homo- or co-polymers of vinyl pyrrolidone, ethylene glycol, and/or vinyl alcohol. In some embodiments, the hydrophilic polymer can be either poly(N-isopropylacrylamide) (PNIPAM), polyacrylamide (PAM), poly(-oxazoline) and polyethylenimine (PEI), poly(acrylic acid), polymethacrylate and other acrylic polymers, poly(ethylene glycol) and poly(ethylene oxide), poly(vinyl alcohol) (PVA) and Ccpolymers, poly(vinylpyrrolidone) (PVP) and copolymers, polyelectrolyte, cucurbit[n]uril hydrate, as well as co-block polymers. In certain embodiments, the hydrophilic polymer includes a second hydrophilic polymer. In certain embodiments, the hydrophilic polymer includes one of poly(vinyl pyrrolidone)(PVP), poly(ethylene glycol) (PEG), and poly(vinyl alcohol) (PVA). In certain embodiments, the hydrophilic polymer includes PVP.

The hydrophilic polymer may have any suitable molecular weight, for example, having a mean molecular weight in the range from about 1 kDa to about 10,000 kDa (e.g., from about 1 kDa to about 5,000 kDa, from about 1 kDa to about 1,000 kDa, from about 1 kDa to about 500 kDa, from about 1 kDa to about 100 kDa, from about 1 kDa to about 50 kDa, from about 1 kDa to about 10 kDa, from about 5 kDa to about 10,000 kDa, from about 10 kDa to about 10,000 kDa, from about 50 kDa to about 10,000 kDa, from about 100 kDa to about 10,000 kDa, from about 500 kDa to about 10,000 kDa, from about 1,000 kDa to about 10,000 kDa, from about 5,000 kDa to about 10,000 kDa, from about 10 kDa to about 5,000 kDa, from about 50 kDa to about 1,000 kDa, from about 100 kDa to about 500 kDa).

In certain embodiments, the hydrophilic polymer has a mean molecular weight in the range from about 1 kDa to about 1,000 kDa (e.g., from about 1 kDa to about 50 kDa, from about 50 kDa to about 500 kDa, from about 500 kDa to about 1,000 kDa).

In certain embodiments, the hydrophilic polymer has a mean molecular weight in the range from about 1 kDa to about 100 kDa (e.g., from about 1 kDa to about 10 kDa, from about 10 kDa to about 50 kDa, from about 50 kDa to about 100 kDa).

The hydrophilic polymer may be present in the composition at any suitable concentration in aqueous composition, for example, from about 2 w/v % to about 10 w/v % (e.g., from about 2 w/v % to about 9 w/v %, from about 2 w/v % to about 8 w/v %, from about 2 w/v % to about 7 w/v %, from about 2 w/v % to about 6 w/v %, from about 2 w/v % to about 5 w/v %, from about 3 w/v % to about 10 w/v %, from about 4 w/v % to about 10 w/v %, from about 5 w/v % to about 10 w/v %, from about 6 w/v % to about 10 w/v %, from about 3 w/v % to about 8 w/v %, from about 4 w/v % to about 9 w/v %). In certain embodiments, the hydrophilic polymer is present in the composition at a concentration from about 2 w/v % to about 7 w/v % (e.g., from about 2 w/v % to about 3.5 w/v %, from about 3.5 w/v % to about 5 w/v %, from about 5 w/v % to about 7 w/v %).

The rubber glove elastomeric material may or may not include vulcanized or non-vulcanized. Typically, the rubber substrate is hydrophobic. The term rubber may compose of the following or a blend of the following items: natural rubber latex (NRL), synthetic latex, neoprene (polychloroprene), nitrile (carboxylated butadiene-acrylonitrile), vinyl(polyvinyl chloride) (PVC), styrene-butadiene rubber (SBR), styrene ethylene butadiene styrene (SEBS), polyurethane, polyisoprene, and other butadiene-based synthetic rubber-based materials. The latex polymer microparticles may be natural latex or synthetic latex. Synthetic rubber latex may be synthetized from, for example, nitrile, butadiene, styrene-butadiene, chloroprene, isobutylene, or co-polymers thereof.

The rubber suspension polymer microparticles may be present in the composition at any suitable concentration in aqueous composition, for example, from about 20 w/v % to about 65 w/v % (e.g., from about 30 w/v % to about 65 w/v %, from about 40 w/v % to about 65 w/v %, from about 50 w/v % to about 65 w/v %, from about 20 w/v % to about 50 w/v %, from about 20 w/v % to about 40 w/v %, from about 20 w/v % to about 30 w/v %). The weight ratio of the hydrophilic polymer to the latex polymer microparticles in aqueous composition may be any suitable value, for example, in the range from about 1:1 to about 1:3 (e.g., about 1:1 to about 1:2.5, about 1:1 to about 1:2, about 1:1 to about 1:1.5, about 1:1.5 to about 1:3, about 1:2 to about 1:3, about 1:2.5 to about 1:3). A blend of rubber suspension can also be considered of more than one kind of rubber.

The aqueous composition is preferably a well-mixed and stable suspension.

The aqueous composition is preferably a viscous aqueous composition, for example, with a viscosity in the range from about 10 cP to about 10¹⁰ cP (e.g., from about 10 cP to about 10² cP, from about 10 cP to about 10⁴ cP, from about 10 cP to about 10⁶ cP, from about 10 cP to about 10⁸ cP, from about 10² cP to about 10¹⁰ cP, from about 10⁴ cP to about 10¹⁰ cP, from about 10⁶ cP to about 10¹⁰ cP, from about 10⁸ cP to about 10¹⁰ cP, from about 10² cP to about 10⁶ cP, from about 10⁴ cP to about 10⁸ cP).

The compositions disclosed herein may further include additives that impact chemical and/or physical properties of the composition, such as vulcanizing and/or accelerating agents. Any suitable vulcanizing agents (e.g., diisopropyl xanthogen polysulfide, sulfur, or ammonia) may be used. Any suitable accelerating agents (e.g., zinc-N-diethyl-dithio-carbomate, zinc-N-dibutyl-dithio-carbomate, or ammonia) may be used.

The compositions disclosed herein may further include one or more of antimicrobials, antifungals, antivirals, vitamins, colors, pigments, and antibiotics.

In another aspect, the invention generally relates to a composition formed by mixing: a first aqueous solution of a hydrophilic polymer with a concentration in the range from about 1 w/v % to about 20 w/v %; and a second aqueous suspension of latex polymer microparticles with a concentration in the range from about 20 w/v % to about 65 w/v %. The hydrophilic polymer has a mean molecular weight in the range from about 1 kDa to about 1,000 kDa. The volume ratio of the first aqueous solution to the second aqueous suspension is in the range from about 1:1 to about 1:3.

In certain embodiments, the hydrophilic polymer at a concentration in the composition from about 2 w/v % to about 7 w/v % (e.g., from about 2 w/v % to about 3.5 w/v %, from about 3.5 w/v % to about 5 w/v %, from about 5 w/v % to about 7 w/v %) and the latex polymer microparticles at a concentration from about 20 w/v % to about 65 w/v % (e.g., e.g., from about 30 w/v % to about 65 w/v %, from about 40 w/v % to about 65 w/v %, from about 50 w/v % to about 65 w/v %, from about 20 w/v % to about 50 w/v %, from about 20 w/v % to about 40 w/v %, from about 20 w/v % to about 30 w/v %).

In certain embodiments, the weight ratio of the hydrophilic polymer to the rubber polymer is in the range from about 1:1 to about 10:1 (e.g., from about 1:1 to about 7:1, from about 1:1 to about 5:1, from about 1:1 to about 3:1, from about 3:1 to about 10:1, from about 5:1 to about 10:1, from about 7:1 to about 10:1).

In certain embodiments, the weight ratio of the hydrophilic polymer to the rubber polymer is in the range from about 1:1 to about 5:1 (e.g., from about 1:1 to about 3:1, from about 1:1 to about 3:1, from about 5:1 to about 5:1, from about 3:1 to about 5:1).

In certain embodiments, the hydrophilic polymer includes a second hydrophilic polymer.

In certain embodiments, the composition includes one or more of vulcanizing agents. In certain embodiments, the composition includes one or more of accelerating agents, preservatives, vulcanization reagents, pigments and/or whiteners or like reagents should also be considered.

In certain embodiments, one or more of vulcanizing agents or the one or more of accelerating agents are present in the first aqueous solution a hydrophilic polymer.

In certain embodiments, one or more of vulcanizing agents or the one or more of accelerating agents are present in the second aqueous suspension of rubber suspension polymer micro particles.

In certain embodiments, the vulcanizing agents are selected from the group consisting of: diisopropyl xanthogen polysulfide, sulfur and ammonia.

In certain embodiments, the accelerating agents are selected from the group consisting of: zinc-N-diethyl-dithio-carbomate, zinc-N-dibutyl-dithio-carbomate and ammonia.

In certain embodiments, one or more of antimicrobials, antifungals, antivirals, vitamins, colors, or antibiotics may be present in the first aqueous solution a hydrophilic polymer.

In certain embodiments, one or more of antimicrobials, antifungals, antivirals, vitamins, colors, or antibiotics may be present in the second aqueous suspension of latex polymer microparticles.

In yet another aspect, the invention generally relates to a cured material formed by heating a composition disclosed herein for a time sufficient to form interpenetrating polymer networks of crosslinked hydrophilic polymer and rubber polymer in presence of accelerating reagents, vulcanization reagents, and/or preservatives.

In some embodiments, the HPRB application process onto rubber gloves is performed using the following on-line manufacturing process: Cleaning of the formers, dipping into a coagulant barrel, dipping into a latex barrel, undergoing leaching tanks, beading, vulcanization and heat, second leaching, carbonate slurry, dry heat via oven, and glove stripping. This process can be performed a single time, multiple times, or plurality of times. In certain embodiments HPRB application following a similar process and can replace, be an addition to, or followed by one of these steps. In certain embodiments, the HPRB can be performed off-line after the completion of the process in a separate application step.

In certain embodiments, curing the rubber polymer is conducted at a temperature between about 25° C. to about 200° C. (e.g., between about 35° C. to about 100° C., about 40° C. to about 200° C., about 50° C. to about 200° C., about 60° C. to about 200° C., about 25° C. to about 200° C., about 25° C. to about 50° C., about 25° C. to about 40° C., about 30° C. to about 60° C.) for a time period from about 3 to about 60 minutes (e.g., from about 3 to about 45 minutes, from about 3 to about 30 minutes, from about 3 to about 15 minutes, from about 3 to about 10 minutes, from about 10 to about 30 minutes, from about 15 to about 30 minutes, from about 20 to about 30 minutes, from about 10 to about 25 minutes, from about 10 to about 20 minutes).

In certain embodiments, curing the hydrophilic polymer and a suspension of rubber polymer to generate the HPRB coating is conducted at a temperature between about 25° C. to about 200° C. (e.g., between about 35° C. to about 100° C., about 40° C. to about 200° C., about 50° C. to about 200° C., about 60° C. to about 200° C., about 25° C. to about 200° C., about 25° C. to about 50° C., about 25° C. to about 40° C., about 30° C. to about 60° C.) for a time period from about 3 to about 60 minutes (e.g., from about 3 to about 45 minutes, from about 3 to about 30 minutes, from about 3 to about 15 minutes, from about 3 to about 10 minutes, from about 10 to about 30 minutes, from about 15 to about 30 minutes, from about 20 to about 30 minutes, from about 10 to about 25 minutes, from about 10 to about 20 minutes).

In certain embodiments, the HPRB coating on the glove can include the use of accelerate chemicals. In some embodiments, these accelerators are sulfur based which includes but is not limited to mercaptobenothiazole (MBT), thiurams, carbamates. In some embodiments, there can be more than one accelerator reagents. In other embodiments, accelerators can be included to sensitize the final product or added at the compounding stage. In other embodiments, the HPRB coating can be applied before or after the acceleration step.

In some embodiments, the HPRB rubber glove undergoes a leaching step. In other embodiments, the HPRB can undergo a pre-vulcanization leaching step to remove any residual accelerator chemicals or to reduce proteins. In other embodiments, the HPRB can undergo a post-vulcanization leaching process. In other embodiments, the HPRB coating can be applied before or after the pre- or post-leaching steps in an online or offline process.

In some embodiments, the HPRB coated rubber gloves undergoes a post-dipping process which can composed of chlorination or exposure to like chemicals. The chlorination process can be an on-line or off-line process which can be repeated a plurality of times on both the inside and outside of the HPRB gloves. In some embodiment, the HPRB coated gloves can undergo additional post-dip processing steps that include polymer wash step, tumbling, drying, flipping, or stripping. In other embodiments, the HPRB coating can be applied before or after these steps.

In a preferred embodiment, the HPRB is applied to the inner surface of the rubber glove that will be in contact with skin. In a preferred embodiment, the HPRB coating can be applied to the entire surface of the inner glove. In other embodiments the HPRB coating can be applied only to the inside of the glove only on exclusively the fingers, palm, or sleeve of the gloves. In another embodiment, the HPRB coating can be applied to the outer surface of the glove. In another embodiment, the HPRB coating can be applied to the outer surface of the glove to be turned into side for the final product. In certain embodiments, the HPRB coating is applied on either the compete or portion of the surface on the inner or outside of the rubber glove.

The cured material may be used with any suitable product, for example, selected from the group consisting: surgeon's Glove (including Dental Surgeon's gloves) Microsurgery Glove Orthopedic Surgeon's Glove Autopsy Surgeon's Glove, Specialty/Chemotherapy Surgeon's Glove Radiation Attenuating Surgeon's Gloves, non-medical gloves, embalming gloves, food service gloves, cleaning gloves, examination gloves, rubber gloves for research, and finger cots.

The rubber glove includes: a sheath of an elastomeric material selected from natural or synthetic rubber latex, the sheath having an outer surface and an inner surface; and a layered coating comprising a first or base layer disposed on and adhered to at least a portion of the outer surface of the sheath and a second or top layer disposed on and adhered to at least a portion of the first or base layer. The first or base layer is a cured latex polymer. The second or top layer is a cured blend of a latex polymer and a hydrophilic polymer having a mean molecular weight in the range from about 1 kDa to about 1,000 Da, wherein the weight ratio of the hydrophilic polymer to the latex polymer is in the range from about 10:1 to about 1:10.

In certain embodiments, the HPRB coating results in a multilayered effect. In a preferred embodiment, the first and/or second layer is composed of the rubber elastomeric material of the same as found in the HPRB coating in which its application is followed by the third layer. In another embodiment, the HPRB coating serves as the first layer which is followed by a second and/or third layer of the rubber elastomeric material of the same suspension found in the HPRB coating formulation. In another embodiment, the HPRB coating can be found as an intermediate layer between a first and third outer layer rubber elastomer composed the sample rubber suspension.

In another embodiment, the HPRB coating can be applied in a similar approach in which the different layers are composed of two or more different rubber suspensions to generate the elastomeric material. In other embodiments, the same can be found using a blend of two or more different rubber suspensions to generate the elastomeric material. In a preferred embodiment, the HPRB coating layer is to be composed or partially composed of a rubber suspension that is found in the elastomeric material in improve adhesion to generate the glove material.

As discussed herein, in certain preferred embodiments, the coating application includes applying a layer of the hydrophilic and rubber polymer blend coating formulation over a rubber base layer, which is applied to the rubber product surface. In some embodiments, the sequential hydrophilic coating application and curing process may be repeated a plurality of times. For example, a hydrophilic and rubber polymer coating is applied onto a rubber substrate with a subsequent layer being the hydrophilic and rubber polymer latex. In another embodiment, the subsequent hydrophilic and rubber polymer is applied onto a hydrophilic and latex polymer coating blend that served as the first or base layer. In some embodiments, multiple coatings can be applied dependent on the desired properties to be achieved.

In certain embodiments, the first layer covers substantially the entire outer surface of the rubber glove, the second layer covers substantially all of the first layer, and, if present, the third layer covers substantially all of the second layer surface, and so forth, respectively.

In certain embodiments, when the final coated HPRB rubber glove is in contact with moisture, aqueous fluids, fluids, liquids, water or like-substance, the top layer becomes slippery with durable lubricity.

In certain embodiments, the method further includes: depositing a third or top layer of a composition, comprising a hydrophilic polymer and a suspension of rubber polymer, to at least a portion of the second layer; and curing the third layer of hydrophilic polymer and rubber polymer with exposure to heat for a time sufficient to form a third or top layer.

In certain embodiments, the first layer covers substantially all of the outer surface of the rubber glove, the second layer covers substantially all of the first layer, and, if present, the third layer covers substantially all of the second layer surface, and so forth, respectively.

In certain embodiments, subsequent coating layers (e.g., second or third layers) are applied to the base layer immediately following curing while the base layer is still at elevated temperature (e.g., above 25° C.). In other embodiments, the base layer fully cools to room temperature (25° C.) prior to application of subsequent coating layers.

In certain embodiments, the rubber glove HPRB coating can further include a coating or layer of powders or dusting agents to further improve donning which can be selected from cornstarch, baby powder, potato starch, lycopodium, or talc, applied to the top layer. In other embodiments, the rubber glove can further include another coating or layer having a silicon-based or water-based lubricant applied to the top layer. In other embodiments, the rubber glove coating enhances the glove's donning properties. These reagents include surfactants, nonionic, ionic surfactants. The additional coated layer can include an emollient coating layer of the hydrophilic polymer in addition with glycerol, gluconolactone, D-sorbitol, provitamin-B, chitosan.

In another aspect, the invention generally relates to a method for manufacturing a glove. The method includes: cleaning one or more glove glass or porcelain formers; dipping the one or more glove glass or porcelain formers into a coagulant barrel; dipping the one or more glove glass or porcelain formers into a latex barrel; heating the coated one or more glove glass or porcelain formers; second dipping of the coated one or more glove glass or porcelain formers into the coagulant barrel; second dipping of the coated one or more glove glass or porcelain formers into the latex barrel; second heating of the coated one or more glove glass or porcelain formers; third dipping of the coated one or more glove glass or porcelain formers into a barrel with the composition disclosed herein; third heating of the coated one or more glove glass or porcelain formers to cure the hydrophilic polymer and rubber polymer; beading and vulcanizing the gloves with heat; exposing the gloves to a carbonate slurry; drying the gloves with heat; and stripping to obtain the manufactured gloves.

In certain embodiments, curing the hydrophilic polymer and a suspension of rubber polymer is conducted at a temperature between about 25° C. to about 200° C. (e.g., between about 25° C. to about 180° C., between about 25° C. to about 150° C.) for a time period from about 3 to about 60 minutes (e.g., from about 5 to about 40 minutes, from about 10 to about 50 minutes).

In certain embodiments, the former is composed of glass. In certain embodiments, the former is composed of porcelain.

In certain embodiments, wherein the glove undergoes a leaching step.

In certain embodiments, wherein the glove undergoes a pre-vulcanization leaching step to remove any residual accelerator chemicals or to reduce proteins.

In certain embodiments, the glove undergoes a post-vulcanization leaching process.

In certain embodiments, the glove undergoes a post-dipping process comprised of chlorination.

In certain embodiments, the glove undergoes one or more of protein and/or polymer wash step, tumbling, drying, flipping, or stripping.

In certain embodiments, the coating is applied to the inner surface of the rubber glove that will be in contact with skin.

In certain embodiments, the coating is applied to the outer surface of the glove to be turned into side for the final product.

In certain embodiments, the coating is applied on either the compete or portion of the surface on the inner or outside of the glove.

In yet another aspect, the invention generally relates to a glove manufactured by a method disclosed herein.

In certain embodiments, the average pore size in the glove of the present invention is in the range of 0 to about 5 μm (e.g., 0.1 μm to about 5 μm, 0.5 μm to about 5 μm, 1 μm to about 5 μm, 2 μm to about 5 μm, 0.1 μm to about 2 μm, 0.1 μm to about 1 μm).

In certain embodiments, the glove is one of surgeon's gloves, microsurgery gloves, dental surgeon's gloves, orthopedic surgeon's gloves, autopsy surgeon's gloves, specialty/chemotherapy surgeon's gloves, radiation attenuating surgeon's gloves, non-medical gloves, embalming gloves, food service gloves, cleaning gloves, examination gloves, rubber gloves for research, and finger cots.

In certain embodiments, the glove is sterilized, e.g., by one or more of ethylene oxide sterilization, autoclave, radiation, gamma, and electron beam radiation sterilization.

In certain embodiments, the first or base layer has a thickness in the range from about 0.01 mm to about 1.0 mm (e.g., from about 0.02 mm to about 1.0 mm, from about 0.05 mm to about 1.0 mm, from about 0.1 mm to about 1.0 mm, from about 0.2 mm to about 1.0 mm, from about 0.4 mm to about 1.0 mm, from about 0.6 mm to about 1.0 mm, from about 0.01 mm to about 0.8 mm, from about 0.01 mm to about 0.5 mm, from about 0.01 mm to about 0.2 mm, from about 0.01 mm to about 0.1 mm, from about 0.01 mm to about 0.08 mm, from about 0.01 mm to about 0.05 mm, from about 0.05 mm to about 0.1 mm).

In certain embodiments, the first or base layer has a thickness less than about 1.0 mm (e.g., less than about 0.8 mm, less than about 0.5 mm, less than about 0.4 mm, less than about 0.2 mm, less than about 0.1 mm, less than about 0.05 mm).

In certain embodiments, the second, or the third if present, or top layer has a thickness in the range from about 0.01 mm to about 1.0 mm (e.g., from about 0.02 mm to about 1.0 mm, from about 0.05 mm to about 1.0 mm, from about 0.1 mm to about 1.0 mm, from about 0.2 mm to about 1.0 mm, from about 0.4 mm to about 1.0 mm, from about 0.6 mm to about 1.0 mm, from about 0.01 mm to about 0.8 mm, from about 0.01 mm to about 0.5 mm, from about 0.01 mm to about 0.2 mm, from about 0.01 mm to about 0.1 mm, from about 0.01 mm to about 0.08 mm, from about 0.01 mm to about 0.05 mm, from about 0.05 mm to about 0.1 mm)

In certain embodiments, the second, or the third if present, or top layer has a thickness less than about 1.0 mm (e.g., less than about 0.8 mm, less than about 0.5 mm, less than about 0.4 mm, less than about 0.2 mm, less than about 0.1 mm, less than about 0.05 mm).

The gloves of the invention depict an even, homogeneous, thin coating on the glove surface. In specific embodiments, the preferred thickness of a coated glove is to be a minimum of about 0.10 mm thick. Greater variations in thickness may occur in different areas of the glove if the polymer film used to coat the glove formers has variations. The final coated gloves can have a thickness between about 0.10 to about 0.03 mm. In a preferred embodiment, the resulting polymer coated glove invention has a thickness of a minimum of about 0.08 mm.

The gloves of the invention exhibit a tensile strength of greater than about 1300 psi, preferably greater than about 2600 psi and most preferably, greater than about 3500 psi. The stress at 500% of the gloves of the invention is less than about 3000 psi, preferably less than about 2000 psi and most preferably, less than about 1000 psi. The gloves of the invention have an elongation at break greater than about 200%, preferably greater than about 500%.

The gloves of the invention have a coating with good adhesion properties to the rubber substrate.

The gloves of the invention may exhibit a reduced coefficient of friction (COF) against objects or tissues compared to standard rubber or other gloves that do not possess the inventive coating. The reduction in COF may pertain to objects and tissues including the user's skin, hair, fingernails, and/or hand overall, as well as the soft tissue of a human or animal non-user. The COF may be reduced by a range from 10-90%. The reduced COF may pertain to in-use configurations (e.g. glove sliding against skin) or bench testing configurations (glove sample specimen sliding against glass, metal, synthetic tissue, or natural tissue).

As disclosed herein, suitable additives may be added to assist in achieving one or more desired properties or enhancements in the compositions or products of the inventions.

In certain embodiments, the hydrophilic and latex polymer blend coating formulation, and the coated product, includes one or more antimicrobials such as antifungals, antibacterials, and metallic nanoparticles and/or microparticles that deter microbial growth. Examples of antifungal agents include, but are not limited to, Amphotericin B, lactic acid, sorbic acid, Clotrimazole, Ciclopirox, Carbol-Fushsin Econazole, Enilconazole, Fluconazole, Griseofulvin, Halogropin, Introconazole, Ketoconazole, Miconazole, Mafenide, Naftifine, Nystatin, Oxiconazole, Thiabendazole, Sulconazole, Tolnaftate, Undecylenic acid, Terbinafine, and Silver Sulfadiazine. Additionally, antibiotics and other antimicrobial agents can be selected from the group consisting of bacitracin; the cephalosporins (such as cefazolin, cefadroxil, cephalothin, cephalexin, ceftazidime, ceftriaxone, ceftizoxime, and meropenem); cycloserine; fosfomycin, the penicillins (such as amdinocillin, amoxicillin, ampicillin, azlocillin, benzathine penicillin G, bacamipicillin, carbenicillin, cyclacillin, cloxacillin, dicloxacillin, mezlocillin, methicillin, oxacillin, nafcillin, penicillin G, penicillin V, piperacillin, and ticarcillin); vancomycin; ristocetin; colistin; novobiocin; the polymyxins (such as colistin, colistimathate, and polymyxin B); the aminoglycosides (such as neomycin, amikacin, kanamycin, gentamicin, netilmicin, paromomycin, streptomycin, spectinomycin, and tobramycin), the tetracyclines (such as demeclocycline, do+-xycycline, minocycline, methacycline, and oxytetracycline); carbapenems (such as imipenem); monobactams (such as aztreonam); clindamycin; chloramphenicol; cycloheximide; fucidin; lincomycin; rifampicin; puromycin; other streptomycins; the macrolides (such as erythromycin and oleandomycin); the fluoroquinolones; actinomycin; ethambutol; 5-fluorocytosine; griseofulvin; rifamycins; the sulfonamides (such as sulfacytine, sulfadiazine, sulfisoxazole, sulfamethoxazole, sulfamethizole, and sulfapyridine); and trimethoprim. Other antibacterial agents include, but are not limited to, bismuth containing compounds (such as bismuth aluminate, bismuth subcitrate, bismuth subgalate, and bismuth subsalicylate); nitrofurans (such as nitrofurazone, nitrofurantoin, and furozolidone); metronidazole; tinidazole; nimorazole; zinc-, copper-, or silver-based compounds, particles (micro-or nano-) and benzoic acid.

In certain embodiments, the hydrophilic and latex polymer blend coating formulation, and the coated product, includes one or more antivirals, antiretrovirals, or any like compounds that prevent the spread of viruses. Examples of antiviral agents can include, but are not limited to, adamantine antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors, protease inhibitors, and purine nucleosides. Other agents and drugs can also include Abacavir, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

In certain embodiments, the hydrophilic and latex polymer blend coating formulation, and the coated product, includes one or more vitamins, for example, Vitamins A, C, D, E, K and B, as well as thiamine (B1), riboflavin (B2), niacin (B3), pantothenic acid (B5), pyroxidine(B6), biotin (B7), folate (B9) and cobalamin (B12).

In certain embodiments, the hydrophilic and latex polymer blend coating formulation, and the coated product, includes one or more pigments or colorants, for example, C.I. Pigment Red 48:2 Permanent Carmine, C.I. Pigment Blue 15:2/Copper Phthalocyanine Blue, C.I. Pigment Green 7/Polychloro Copper phthalocyanine Green, C.I. Pigment Yellow 74 Azo Yellow. White pigments that may be used but is not limited to include titanium dioxide and zinc oxide.

In certain embodiments, the hydrophilic aqueous solution or latex polymer blend coating formulation, and the coated product, include other additives suitable for use with a glove product, such as one or more of plasticizers, accelerators, stabilizers, anticoagulants, preservatives, or other compounds can be added to the natural or synthetic latex solution to assist or accelerate the vulcanization process or to improve its mechanical properties. In some embodiments, these additives can include, but are not limited to, ammonia, proteins, nitrosamine, zinc chloride, zinc oxide, stearic acid, antidegradants, plasticizers, sulfur, peroxides, acetic acid, citric acid, formic acid, metallic oxides, potassium laurate, acetoxysilanes, urethane crosslinkers, polychloroprene, ethylene thiourea, or other equivalent accelerators, catalysis, or curatives. In another embodiment, more than one of these additives or compounds can be added into the latex solution. Stabilizers can include are oleates, stearates, alginates, polyacrylates, xanthan gums, caseinates or other nonionic and ionic surfactants. Typical crosslinkers include sulfur or other organic peroxides. Vulcanization activators or accelerators include or chosen from metal oxides, such as magnesium oxide, lead oxide, zinc oxide, mercaptobenzothiazoles and their derivatives, dithiocarbamates and their derivatives, sulfur donors, guanidines and aldehyde-amine reaction products. Antioxidants include hindered arylamines or polymeric hindered phenols. Typical antiozonants, which may be used in the compounding formulation, include paraffinic waxes, microcrystalline waxes and intermediate types of waxes (e.g., combination of both paraffinic and microcrystalline waxes).

The following examples are meant to be illustrative of the practice of the invention, and not limiting in any way.

### Examples

Formulation Preparation and Application onto Premade Latex Rubber Gloves

Commercial disposable latex gloves (non-coated and non-lubricated) were prepared by turning the glove inside out, washing off any powder off the glove surface, and placing the glove onto a glass former in the shape of the glove. Next the former with the glove is dipped into the coating formulation which was prepared by previously dissolving the hydrophilic polymer (PVP in this example) in water at 5 w/v % which was then mixed with a latex suspension. The former was carefully dipped into the solution and gradually removed at a rate of about 1 in/second. Excess formulation was allowed to drip from the mandrel for about 5 seconds while a thin layer of the coated solution was observed on the surface to ensure the coating was in contact with the glove surface. The coated glove was placed directly into an oven for 15 minutes at about 70° C. to cure the coating onto the glove sample. Samples were removed from the oven and allowed to cool to room temperature before assessing the coating.

The coated glove samples were gently dusted with cornstarch to prevent the rubber from sticking to itself. The final glove samples were then turned inside out once again for the resulting coated surface will be in contact with human skin.

Samples were then accessed in accordance to the following parameters: (A) surface was slippery and wetted when accessed using a wet finger via touch; (B) sample was stretched 3 times at 500% elongation and observed to ensure no delamination of the coating was noted; and, finally (C) ability to maintain lubricity after about 20 rubs when wetted. All polymer coated samples met these requirements.

### Formulation Preparation and Application of Coated Rubber Gloves

Porcelain formers were first washed with warm water and dried prior to forming a latex glove as the base. Formers were dipped into a latex suspension at a minimum of 10 inches of the former before removing the former from solution at a rate of about 1 inch per second. The former was visually observed to ensure the dip resulted an even layer on the porcelain former. The dip was repeated if any inconsistencies or uncoated patches were observed. Excess latex suspension was allowed to drip from the former for about 5 seconds to ensure a thin coating prior towards heat curing the samples in an oven for about 15 minutes at 70° C. to cure the latex. The protocol was repeated a second time to form a double layer latex glove.

After forming the glove, the former is dipped into the coating formulation which was prepared by previously dissolving the hydrophilic polymer (PVP in this example) in water at 5 w/v % which was then mixed with a latex suspension. The former was carefully dipped into the solution and gradually removed at a rate of about 1 in/second. Excess formulation was allowed to drip from the mandrel for about 5 seconds while a thin layer of the coated solution was observed on the surface to ensure the coating was in contact with the glove surface. The coated glove was placed directly into an oven for 15 minutes at about 70° C. to cure the coating onto the glove sample. Samples were removed from the oven and allowed to cool to room temperature before assessing, the coating.

The coated glove samples were gently dusted with cornstarch to prevent the rubber from sticking to itself. The final glove samples were then turned inside out for the resulting coated surface will be in contact with human skin. Samples were then accessed in accordance to the following parameters: (A) surface was slippery and wetted when accessed using a wet finger via touch; (B) sample was stretched 3 times at 500% elongation and observed to ensure no delamination of the coating was noted; and, finally (C) ability to maintain lubricity after about 20 rubs when wetted. All polymer coated samples met these requirements.

### SEM Analysis of Coated Rubber Materials

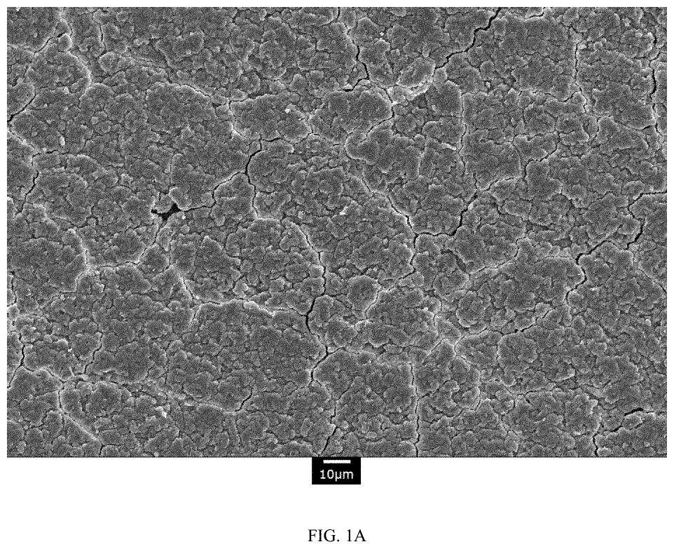

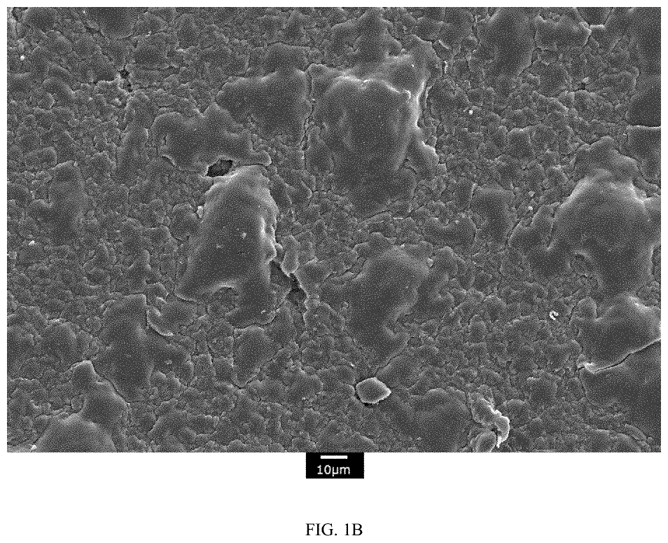

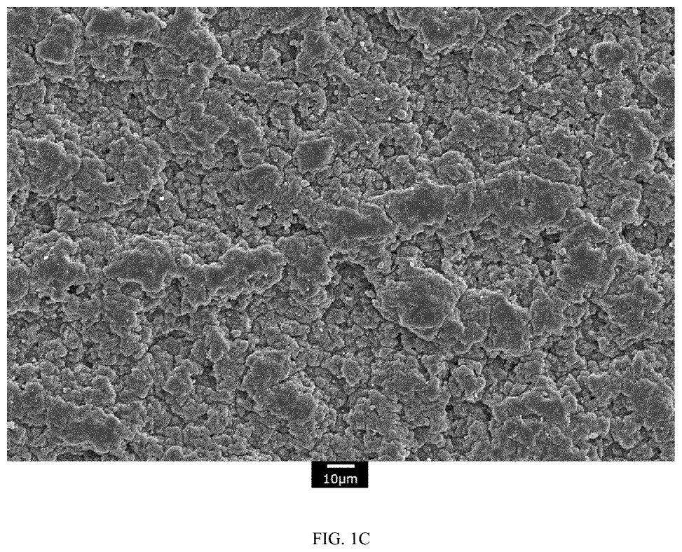

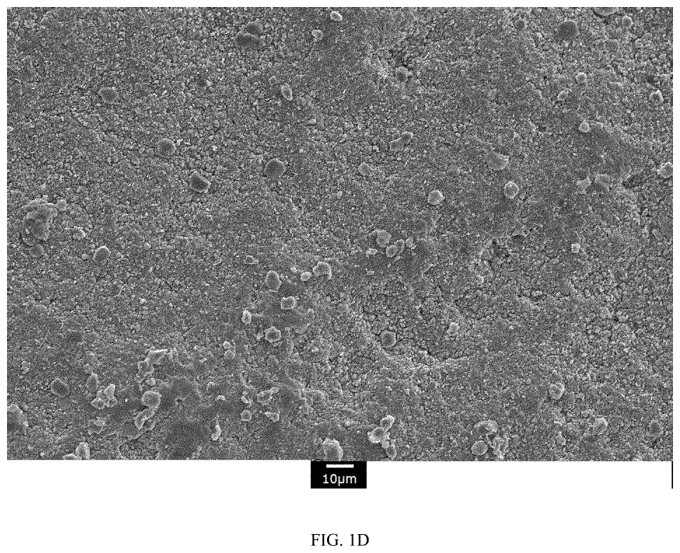

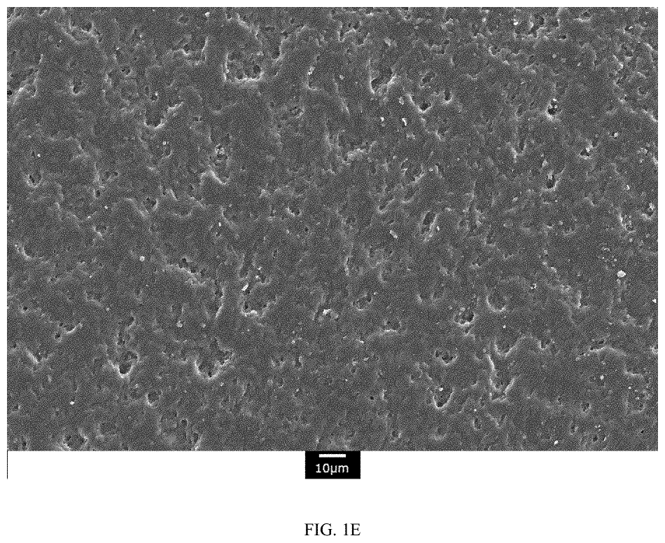

Scanning electron microscopy (SEM) images at 500× magnification of non-coated and coated latex disposable glove samples were performed to examine and compare the coated surface properties between the following samples: non-coated gloves, commercially available polymer coated gloves (Biogel®), nitrile-coated gloves, powdered coated gloves. Results are noted in FIG. 1. The Polymer coated sample depicted an even and consistent coated surface which was comparable to the other glove samples.

### Thickness Testing of Coated Rubber Materials

Thickness measurements were performed using a digital micrometer on 3 samples per each batch. Measurements were taken on the palm of the gloves which were averaged and reported in the table below at an n=3. The polymer coated samples were prepared using latex disposable gloves which was compared to commercially available gloves, both with and without a coating, in which its surface has been treated for improve donning. Samples included the following: non-coated gloves, commercially available polymer coated gloves (Biogel®), nitrile-coated gloves, powdered coated gloves. Results are noted in the following, table. The polymer coated samples appear to a similar coating thickness and good reproducibility compared to the other commercially available gloves.

TABLE 1

| | Latex | Commercial | Nitrile- | Powder- | Polymer |
|---|---|---|---|---|---|
| | Control | Polymer Coated | Coated | Coated | Coated |
| | (Control) | Sample (Control) | (Control) | (Control) | Sample |
| | Sample 1 (inches) | | | | |
| Average | 0.0034 | 0.0075 | 0.0074 | 0.0033 | 0.0061 |
| Std | 0.0004 | 0.0003 | 0.0002 | 0.0001 | 0.0002 |
| | Sample 2 (inches) | | | | |
| Average | 0.0037 | 0.0076 | 0.0072 | 0.0033 | 0.0046 |
| Std | 0.0002 | 0.0002 | 0.0003 | 0.0002 | 0.0023 |

### Adhesion Coating Testing

The adhesion of the coated substrate was compared to 4 samples by evaluating its ability to stay on the rubber surface. In summary, the finger and palm areas of the coated glove sectioned and stretched to about 500% its length 5 times. The coated surface was then laid flat on a hard surface and rubbed vigorously using the thumb for 5 seconds. Next the surfaces were examined visually for any residual coated flakes, or powdery substance to indicate delamination or removal of the coating. The coating adhesion was rated qualitatively from 1 to 5, with 1 being the worst where the entire coating was removed from the rubber substrate and with 5 being the best case which was identified as no flaking occurred along with the coating staying intact and adhered onto the rubber substrate.

The polymer coated samples were prepared using latex disposable gloves which was compared to commercially available gloves, both with and without a coating, in which its surface has been treated for improve donning. Samples included the following: non-coated gloves, commercially polymer coated gloves (Biogel®), nitrile-coated gloves, powdered coated gloves. Results are noted in the following, table. The polymer coated samples appear to have good adhesion onto the latex substrates under a comparable performance to current commercially available gloves.

TABLE 2

| | Latex | Commercial | Nitrile- | Powder- | Polymer |
|---|---|---|---|---|---|
| | Control | Polymer Coated | Coated | Coated | Coated |
| | (Control) | Sample (Control) | (Control) | (Control) | Sample |
| 1 | | | | | |
| 2 | | | | | |
| 3 | | | | | |
| 4 | | | | | |
| 5 | X | x | x | x | X |

### Tensile Testing

Tensile testing was performed on an Instron 5944 Universal Tester equipped with a 100 N load coll. Testing was conducted according to ASTM D412-2016. 0.25″-wide rectangular latex strips were mounted via pneumatic grips with elastic polymeric grip faces. Initial gauge length was 1″. The machine crosshead was displaced at a rate of $0.333 mm/s while specimens under tension were monitored to ensure that no slippage occurred. Tension proceeded until sample breakage. Parameters extracted included stress at break (tensile strength), strain at break, and stress at 500% elongation. Testing for the polymer coated samples was conducted under both dry and wetted (with water) conditions.

TABLE 3

| | Latex | Commercial | | |
|---|---|---|---|---|
| | Control | Polymer Coated | Polymer Coated | Polymer Coated |
| | (Control) | Sample (Control) | Sample (dry) | Sample (wetted) |
| Stress at break | 33.1 ± 5.7 MPa | 26.5 ± 5.9 MPa | 21.2 ± 3.3 MPa | 22.6 ± 3.6 MPa |
| (tensile strength) | | | | |
| Strain at break | 906 ± 44% | 1306 ± 84% | 979 ± 50% | 1037 ± 44% |
| Stress at 500% | 8.1 ± 1.6 MPa | 3.1 ± 0.5 MPa | 7.1 ± 2.0 MPa | 5.0 ± 0.8 MPa |
| elongation | | | | |

Mean ± standard deviation, n = 5.

### Coefficient of Friction Testing

Coefficient of friction measurements were determined using a standardized test method for Static and Kinetic Coefficients of Friction of Plastic Film and Sheeting via. ASTM 1894-1.995. The protocol used measures static and kinetic starting and sliding friction of plastic film and sheeting when sliding over itself or other substances. Tests were conducted using an AMETEK TCD225 Digital Force Tester wider both dry and wetted (with water) conditions. A sled containing a 2.5″×2.5″ flat bottom surface with the latex sample adhered to it using double-sided foam tape was slid at a speed of 150 min/min. for 90 ram against a larger glass counter surface.

TABLE 4

| | | | Polymer | Nitrile- | Powder | Polymer |
|---|---|---|---|---|---|---|
| | | Latex Control | Coated | Coated | Coated | Coated |
| | | (Control) | (Control) | (Control) | (Control) | Sample |
| Static | Dry | 0.220 ± 0.047 | 0.144 ± 0.040 | 0.224 ± 0.027 | 0.363 ± 0.062 | 3.471 ± 0.385 |
| COF | Wetted | 0.174 ± 0.027 | 0.310 ± 0.095 | 0.133 ± 0.013 | 0.150 ± 0.010 | 0.129 ± 0.029 |
| Kinetic | Dry | 0.313 ± 0.035 | 0.146 ± 0.043 | 0.266 ± 0.026 | 0.449 ± 0.067 | 2.872 ± 0.676 |
| COF | Wetted | 0.177 ± 0.033 | 0.452 ± 0.138 | 0.152 ± 0.032 | 0.199 ± 0.022 | 0.191 ± 0.041 |

Static COF. Mean ± standard deviation, n = 5.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An aqueous composition, comprising:
a hydrophilic polymer; and
a suspension of rubber polymer microparticles,
wherein
the hydrophilic polymer has a mean molecular weight in the range from about 1 kDa to about 300,000 kDa and is present in the composition at a concentration from about 0.1 w/v % to about 10 w/v %,
the rubber polymer microparticles are present in the composition at a concentration from about 20 w/v % to about 60 w/v %, and
the weight ratio of the hydrophilic polymer to the rubber polymer microparticles is in the range from about 1:1 to about 10:1.

2-20. (canceled)

21. A composition formed by mixing:
a first aqueous solution of a hydrophilic polymer with a concentration in the range from about 1 w/v % to about 20 w/v %; and
a second aqueous suspension of rubber polymer microparticles with a concentration in the range from about 20 w/v % to about 65 w/v %, wherein
the hydrophilic polymer has a mean molecular weight in the range from about 1 kDa to about 1,000 kDa; and
the volume ratio of the first aqueous solution to the second aqueous suspension is in the range from about 1:1 to about 1:3.

22-36. (canceled)

37. A cured material formed by heating a composition according to claim 1 for a time sufficient to form interpenetrating polymer networks of crosslinked hydrophilic polymer and rubber polymer, wherein heating a composition is performed in the presence of one or more of accelerating reagents, vulcanization reagents, and preservatives, wherein the material is a coating forming a surface of a glove.

38-39. (canceled)

40. A method for manufacturing a glove, comprising:
cleaning one or more glove glass or porcelain formers;
dipping the one or more glove glass or porcelain formers into a coagulant barrel;
dipping the one or more glove glass or porcelain formers into a latex barrel;
heating the coated one or more glove glass or porcelain formers;
second dipping of the coated one or more glove glass or porcelain formers into the coagulant barrel;
second dipping of the coated one or more glove glass or porcelain formers into the latex barrel;
second heating of the coated one or more glove glass or porcelain formers;
third dipping of the coated one or more glove glass or porcelain formers into a barrel with the composition according to any of claims 1-36;
third heating of the coated one or more glove glass or porcelain formers to cure the hydrophilic polymer and rubber polymer;
beading and vulcanizing the gloves with heat;
exposing the gloves to a carbonate slurry;
drying the gloves with heat; and
stripping to obtain the manufactured gloves.

41. The method of claim 40, wherein the one or more glove formers are glass formers.

42. The method of claim 40, wherein the one or more glove formers are porcelain formers.

43. The method of claim 40, further comprising sterilizing the glove, wherein the sterilization procedure comprises one or more of ethylene oxide sterilization, autoclave, radiation, gamma, and electron beam radiation sterilization.

44. (canceled)

45. The method of claim 40, wherein curing the hydrophilic polymer and a suspension of rubber polymer is conducted at a temperature between about 25° C. to about 200° C. for a time period from about 3 to about 60 minutes.

46. The method of claim 40, wherein the glove undergoes a leaching step.

47. The method of claim 46, wherein the glove undergoes a pre-vulcanization leaching step to remove any residual accelerator chemicals or to reduce proteins.

48. The method of claim 46, wherein the glove undergoes a post-vulcanization leaching process.

49. The method of claim 40, wherein the glove undergoes a post-dipping process comprised of chlorination.

50. The method of claim 40, wherein the glove undergoes one or more of polymer wash step, tumbling, drying, flipping and stripping.

51. The method of claim 40, wherein the coating is applied to the inner surface of the rubber glove that will be in contact with skin.

52. The method of claim 40, wherein the coating is applied to the outer surface of the glove to be turned into side for the final product.

53. The method of claim 40, wherein the coating is applied on either the compete or portion of the surface on the inner or outside of the glove.

54. A glove manufactured according to the method of claim 40.

55. The glove of claim 54, wherein the glove is characterized by an average pore size in the range of about 0.1 μm to about 5 μm.

56. The glove of claim 54, wherein the glove is one of surgeon's gloves, microsurgery gloves, dental surgeon's gloves, orthopedic surgeon's gloves, autopsy surgeon's gloves, specialty/chemotherapy surgeon's gloves, radiation attenuating surgeon's gloves, non-medical gloves, embalming gloves, food service gloves, cleaning gloves, examination gloves, rubber gloves for research, and finger cots.

57. The glove of claim 54, wherein the glove is sterilized.
